(19) United States
(12) Patent Application Publication
(10) Pub. No.: US 2026/0108280 A1
(43) Pub. Date: Apr. 23, 2026

(54) AUTOMATIC SCREW IMPLANTATION SYSTEM

(71) Applicant: Point Robotics Medtech Inc., Hsinchu County (TW)

(72) Inventors: SHIH-YUAN CHEN, Hsinchu County (TW); Yun-Shuo Shi, Hsinchu County (TW); Yong-Hou Tian, Hsinchu County (TW)

(21) Appl. No.: 19/021,206

(22) Filed: Jan. 15, 2025

(30) Foreign Application Priority Data
Oct. 17, 2024 (TW) 113139455

(51) Int. Cl. A61B 17/70; A61B 17/00; A61B 17/86; A61B 34/10; A61B 34/32; A61B 90/00

(52) U.S. Cl. CPC A61B 17/7082; A61B 34/32; A61B 2017/00398; A61B 17/864; A61B 2034/107; A61B 2090/066

(57) ABSTRACT

An automatic screw implantation system includes a screw implantation device. The screw implantation device includes a power transmission assembly, a guide pin, and a motor. The power transmission assembly is used to be connected to a hollow pedicle screw. The guide pin passes through and is arranged coaxially with the hollow pedicle screw. The motor is dynamically coupled to the power transmission assembly and the guide pin. The motor is configured for driving the guide pin to advance and simultaneously spin at a first rotational speed and a first torque. The motor is configured for driving the power transmission assembly to control the hollow pedicle screw to spin at a second rotational speed and a second torque. The second rotational speed is smaller than the first rotational speed, and the second torque is greater than the first torque.

### CROSS-REFERENCE TO RELATED PATENT APPLICATION

This application claims the benefit of priority to Taiwan Patent Application No. 113139455, filed on Oct. 17, 2024. The entire content of the above identified application is incorporated herein by reference.

### FIELD OF THE DISCLOSURE

The present disclosure relates to a screw implantation system, and more particularly to an automatic screw implantation system.

### BACKGROUND OF THE DISCLOSURE

Spinal fusion surgery is a common procedure for treating low back pain, which involves repeating several steps: making a small incision on the skin surface, creating a guide hole using instruments such as a trocar or bone drill, and then inserting a guide wire. Afterward, a hollow pedicle screw is locked into the spine along the guide wire to complete the implantation and stabilization of the screws.

However, the existing methods for implanting the pedicle screws require complicated surgical steps and exchange of instruments. Additionally, when placing a pedicle screw at the guide hole along the guide wire, the position of the screw may easily shift from the guide hole, resulting in a final implantation position that differs from the initially predetermined location.

Therefore, how to overcome the above-mentioned problem through an improvement in structural design has become an important issue to be addressed in the related art.

### SUMMARY OF THE DISCLOSURE

In response to the aforementioned technical inadequacy, the present disclosure provides an automatic screw implantation system to address the issue of existing techniques for implanting pedicle screws, which require complicated surgical steps and exchange of instruments, thereby affecting efficiency and accuracy of the screw implantation.

In order to solve the above-mentioned problems, one of the technical aspects adopted by the present disclosure is to provide an automatic screw implantation system. The automatic screw implantation system includes a screw implantation device. The screw implantation device includes a motor, a power transmission assembly, and a guide pin. The power transmission assembly is used to be connected to a hollow pedicle screw. The guide pin passes through and is arranged coaxially with the hollow pedicle screw. The motor is dynamically coupled to the power transmission assembly and the guide pin. The motor is configured for driving the guide pin to advance and simultaneously spin at a first rotational speed and a first torque. The motor is configured for driving the power transmission assembly to control the hollow pedicle screw to spin at a second rotational speed and a second torque. The second rotational speed is smaller than the first rotational speed, and the second torque is greater than the first torque.

Therefore, in the automatic screw implantation system provided by the present disclosure, the motor drives the guide pin to spin at a high rotational speed and a low torque for drilling a guide hole, and the motor then drives the power transmission assembly to screw the hollow pedicle screw into the guide hole at a low rotational speed and a high torque. Therefore, the automatic screw implantation system combines the functions of drilling and screwing. As a result, spinal fusion surgery can be completed without changing surgical instruments, thereby simplifying the surgery procedures and enhancing efficiency and accuracy of the surgery.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an automatic screw implantation system according to an embodiment of the present disclosure;

FIG. 2 is a functional block diagram of the automatic screw implantation system according to the embodiment of the present disclosure;

FIG. 3 is a schematic view of a screw implantation device according to the embodiment of the present disclosure;

FIG. 4 is a partial schematic enlarged view of the screw implantation device according to the embodiment of the present disclosure;

FIG. 5 is a schematic view of a guide pin and a hollow pedicle screw in a drilling process according to the embodiment of the present disclosure;

FIG. 6 is a schematic view of the guide pin and the hollow pedicle screw in a locking process according to the embodiment of the present disclosure;

FIG. 7 is a schematic view of steps S1 to S9 of a method for operating the automatic screw implantation system according to the embodiment of the present disclosure; and

FIG. 8 is a schematic view of steps S11 to S13 of the method for operating the automatic screw implantation system according to the embodiment of the present disclosure.

### DETAILED DESCRIPTION OF THE EXEMPLARY EMBODIMENTS

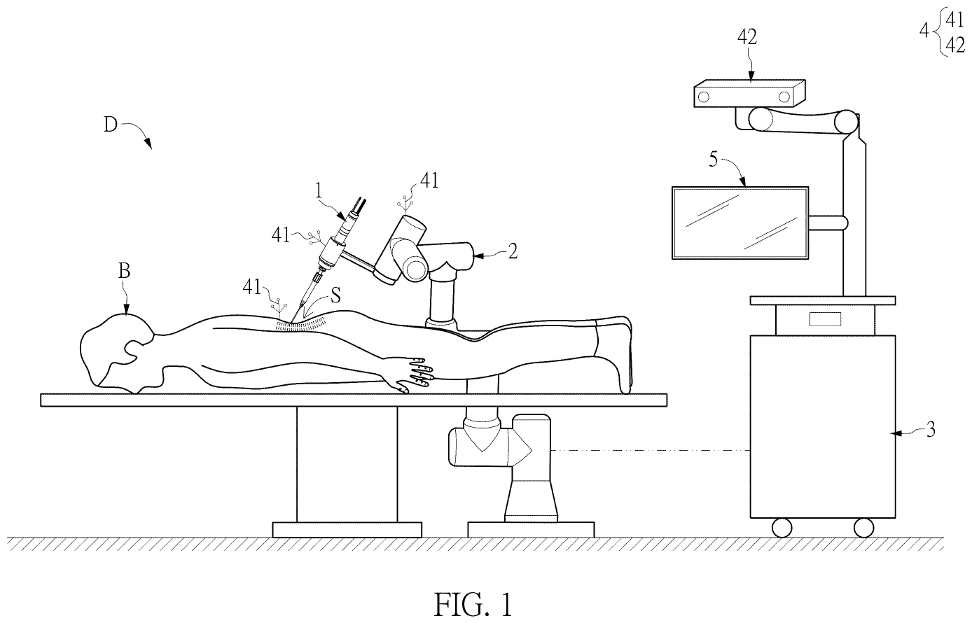

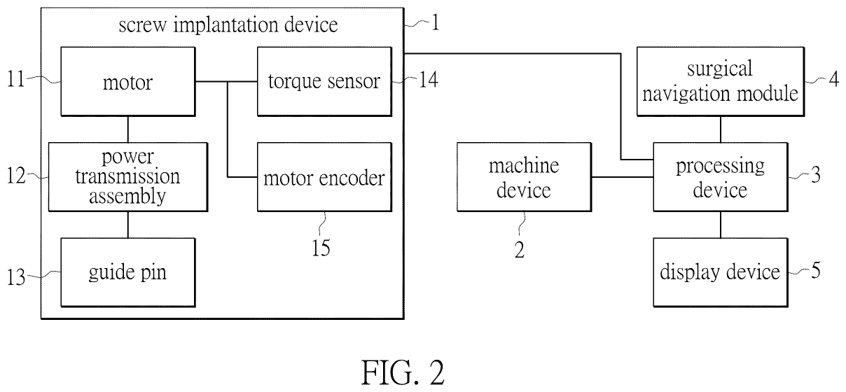

Reference is made to FIG. 1 and FIG. 2. An embodiment of the present disclosure provides an automatic screw implantation system D. The automatic screw implantation system D includes a screw implantation device 1, a robot device 2, and a processing device 3. The robot device 2 is connected to the screw implantation device 1. The processing device 3 is electrically connected to the robot device 2 and is used to control operation of the robot device 2.

For example, the robot device 2 can be a mechanical arm that can move with multiple degrees of freedom. Through an adapter, the robot device 2 can grip surgical instruments, such as the screw implantation device 1. The processing device 3 can includes a processor and a memory, but the present disclosure is not limited thereto. The processor can be, for example, a programmable logic control circuit, an integrated circuit of a microprocessor circuit or a micro-control circuit, or a central processing unit. The memory can be, for example, a random access memory (RAM), a read only memory (ROM), a flash memory, a hard disk or other storage devices that can be used to store data.

The screw implantation device 1 includes a motor 11, a power transmission assembly 12, and a guide pin 13. The processing device 3 is electrically connected to the screw implantation device 1 and is used to control the motor 11. The power transmission assembly 12 is connected between the motor 11 and the guide pin 13. The power transmission assembly 12 and the guide pin 13 are dynamically coupled to the motor 11.

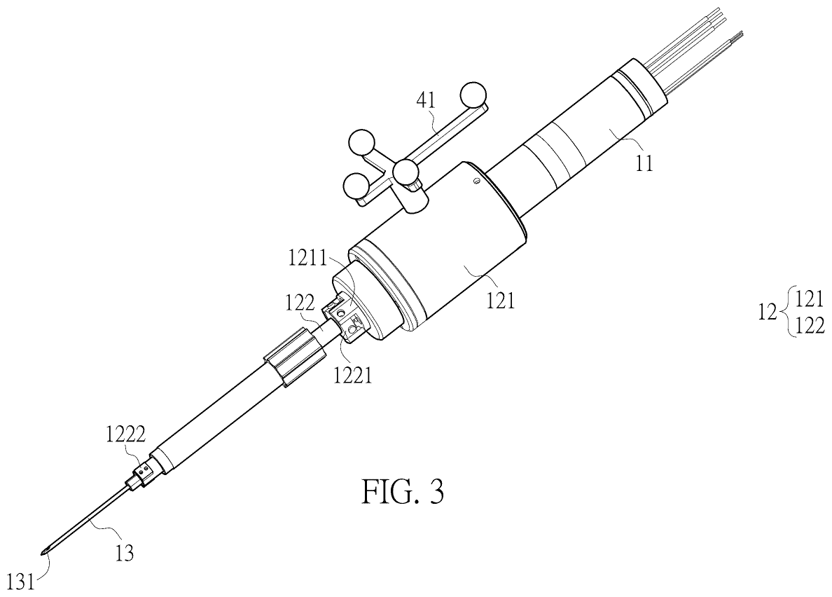

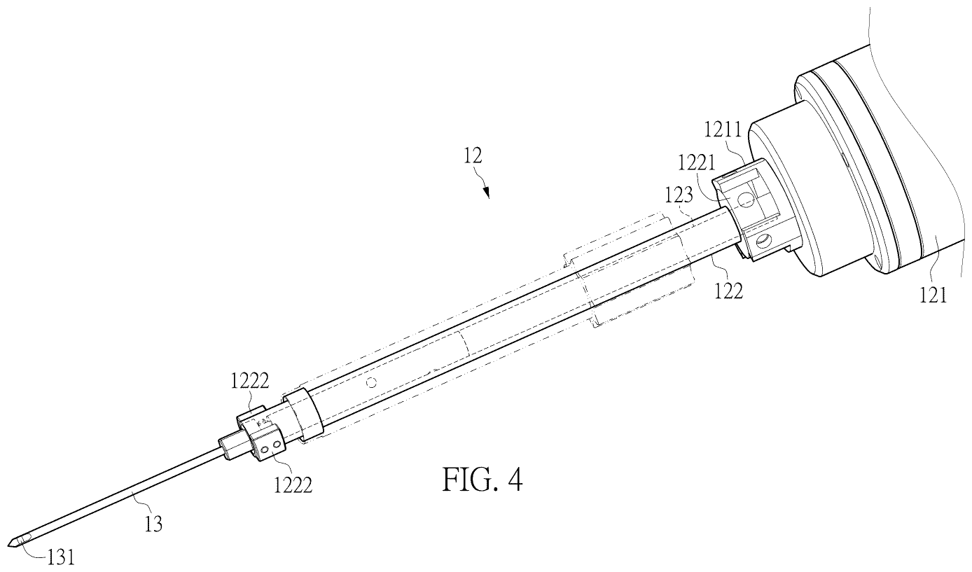

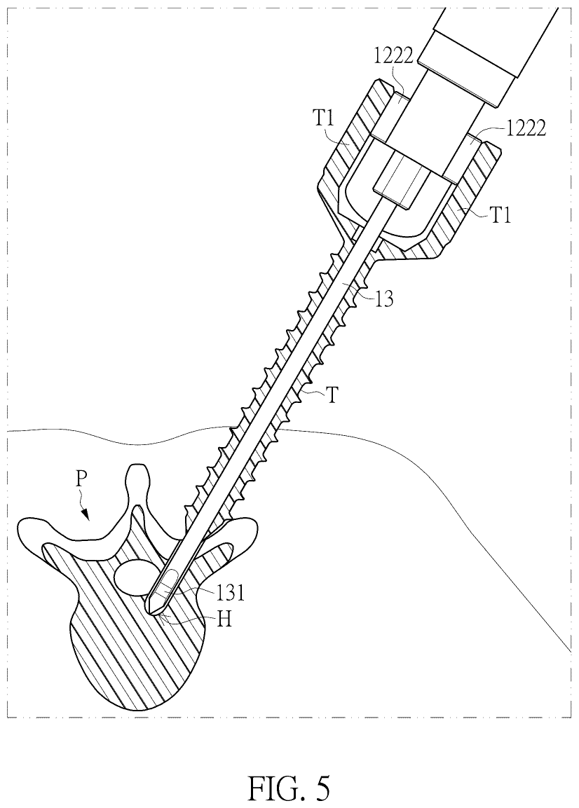

Reference is made to FIG. 3 and FIG. 4. For example, the power transmission assembly 12 includes a coupling 121 and a linkage assembly that is connected to the coupling 121. The linkage assembly includes a first rod 122 and a second rod 123 that is coaxial with the first rod 122. The first rod 122 is sleeved around the second rod 123, and each of the first rod 122 and the second rod 123 operates independently. The coupling 121 is connected to the motor 11. The motor 11 transmits power to the first rod 122 and the second rod 123 through the coupling 121. One end of the second rod 123 is connected to the coupling 121, and the other end of the second rod 123 is connected to the guide pin 13. The guide pin 13 is also called puncture needle or K-pin. Furthermore, as shown in FIG. 5, the power transmission assembly 12 is used to be connected to a hollow pedicle screw T. The guide pin 13 passes through and is arranged being coaxial with the hollow pedicle screw T. More specifically, the guide pin 13 and the hollow pedicle screw T are spaced apart from and do not interfere with each other.

Reference is made to FIG. 4 and FIG. 5. One end of the first rod 122 includes a first connecting portion 1221, and the other end of the first rod 122 includes two second connecting portions 1222. The coupling 121 includes a connecting portion 1211. Through the connecting portion 1211, the coupling 121 is connected to the first connecting portion 1221 of the first rod 122. An end of the hollow pedicle screw T includes a U-shaped structure. After the guide pin 13 passes through the hollow pedicle screw T, two ear portions T1 of the U-shaped structure are respectively connected to the two second connecting portions 1222 of the first rod 122, so that the hollow pedicle screw T is fixed to the screw implantation device 1. For example, the U-shaped structure of the hollow pedicle screw T can be connected to the second connecting portions 1222 of the first rod 122 by snapping, fittingly engaging or fastening, but the present disclosure is not limited thereto.

Reference is further made to FIG. 1 and FIG. 2. The automatic screw implantation system D further includes a surgical navigation module 4. The surgical navigation module 4 includes a plurality of navigation markers 41 and an optical tracking sensor 42. The plurality of navigation markers 41 are distributed on the robot device 2 and the screw implantation device 1, and near a target site (surgical site P of a patient B). Each of the navigation markers 41 includes a dynamic reference frame (DRF) and a plurality of optical elements that are disposed on the dynamic reference frame. The optical elements can be, for example, reflective balls or markers that can generate perceptible signals. The optical tracking sensor 42 is electrically connected to the processing device 3. The plurality of navigation markers 41 can serve as spatial reference points for establishing a spatial coordinate system, while the optical tracking sensor 42 is capable of sensing, detecting, and recording coordinate positions of the plurality of optical elements on the navigation markers 41, and transmitting the information (i.e., the coordinate positions) to the processing device 3 for appropriate calculations and/or storage.

The automatic screw implantation system D further includes a display device 5 that is electrically connected to the processing device 3. For example, the display device 5 can include a display screen and a buzzer (not shown in the figures). The processing device 3 acquires images of the area near the surgical site P through the surgical navigation module 4, and integrates pre-operative medical images, such as computed tomography (CT) or magnetic resonance imaging (MRI), to establish a three-dimensional anatomy model of the area near the surgical site P. The constructed three-dimensional anatomy model can be displayed in a navigation interface on the display device 5.

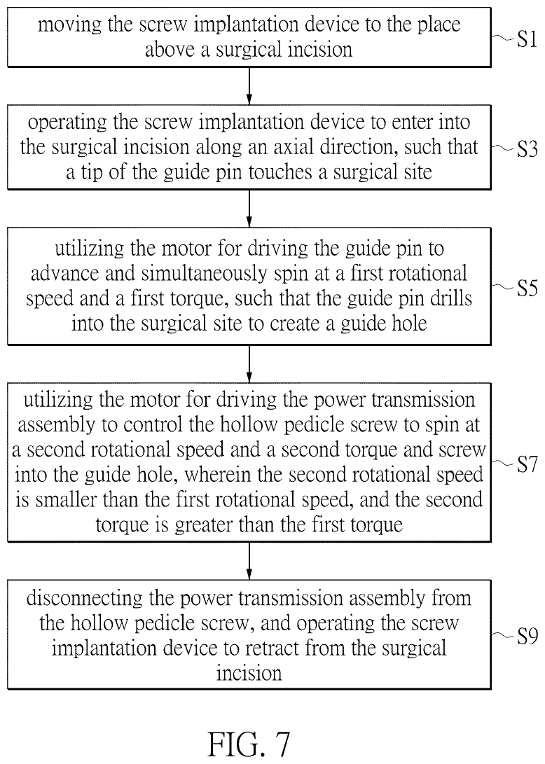

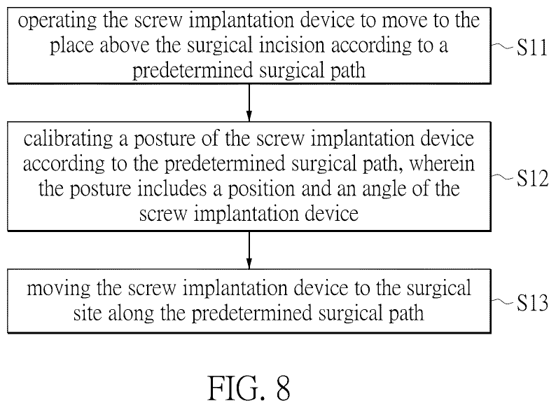

Reference is made to FIG. 7 and FIG. 8. The method for operating the automatic screw implantation system D includes at least following steps:
Step S1: moving the screw implantation device to the place above a surgical incision.
Step S3: operating the screw implantation device to enter into the surgical incision along an axial direction, such that a tip of the guide pin touches a surgical site.
Step S5: utilizing the motor for driving the guide pin to advance and simultaneously spin at a first rotational speed and a first torque, such that the guide pin drills into the surgical site to create a guide hole.
Step S7: utilizing the motor for driving the power transmission assembly to control the hollow pedicle screw to spin at a second rotational speed and a second torque and screw into the guide hole, wherein the second rotational speed is smaller than the first rotational speed, and the second torque is greater than the first torque.
Step S9: disconnecting the power transmission assembly from the hollow pedicle screw, and operating the screw implantation device to retract from the surgical incision.

Moreover, in the method for operating the automatic screw implantation system D, the robot device 2 can further be operated to perform the following steps while operating the screw implantation device (steps S1 and S3):
Step S11: operating the screw implantation device to move to the place above the surgical incision according to a predetermined surgical path.
Step S12: calibrating a posture of the screw implantation device according to the predetermined surgical path, wherein the posture includes a position and an angle of the screw implantation device.
Step S13: moving the screw implantation device to the surgical site along the predetermined surgical path.

Further, the processes for steps S1 to S7, as well as steps S11 to S13 are described in detail. Reference is made to FIGS. 1, 5, and 6. The patient B lies prone on an operating table. When medical personnel prepare to perform surgery, the processing device 3 can plan a predetermined surgical path based on the spatial coordinate system and the constructed three-dimensional anatomy model. Then, the processing device 3 further controls the robot device 2 to calibrate the posture of the screw implantation device 1 according to the predetermined surgical path, such as the position and angle of the screw implantation device 1 relative to the surgical site P (i.e., the pedicle site of the spine). After the medical personnel use a scalpel to create a surgical incision S, the processing device 3 controls the robot device 2 based on the predetermined surgical path to move the screw implantation device 1 to the place above the surgical incision S. After confirming the position of the screw implantation device 1, an axial direction of the screw implantation device 1 is then fixed. The processing device 3 controls the robot device 2 to follow the predetermined surgical path and lower the screw implantation device 1 to the surgical site P. Thus, the medical personnel manually operate the robot device 2 to perform the surgery. Alternatively, the surgery can be automatically performed under controlling the robot device 2 by the processing device 3.

The screw implantation device 1 is operated to move along the axial direction (i.e., the axial direction of the linkage assembly) and enter into the surgical incision S, so that a tip 131 of the guide pin 13 touches the surgical site P. Then, the processing device 3 controls the motor 11 in the screw implantation device 1 to provide power to drive the power transmission assembly 12, so that the guide pin 13 can spin at a high rotational speed. In the meantime, the motor 11 is controlled to transmit power to the second rod 123 through the coupling 121, so that the second rod 123 drives the guide pin 13 to advance toward the surgical site P, and the guide pin 13 drills into the surgical site P in a first rotational direction to create a guide hole H (shown in FIG. 5). Furthermore, the guide pin 13 spins at a first rotational speed and a first torque. Preferably, the first rotational speed is greater than 10,000 rpm, and the first torque is less than 0.5 Nm.

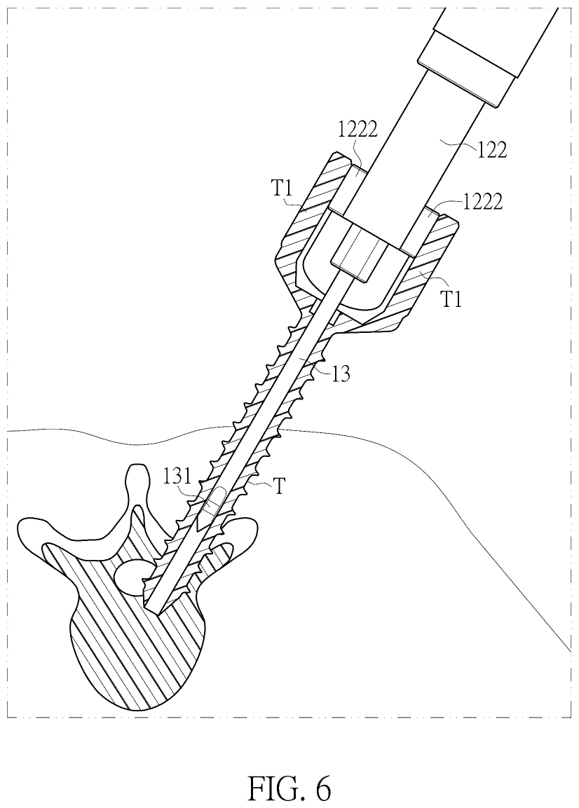

When the guide pin 13 drills into the surgical site P to create the guide hole H, the first rod 122 does not move with the guide pin 13. More specifically, while the guide pin 13 is drilling, the hollow pedicle screw T is sleeved around the guide pin 13 and remains stationary. After the guide hole H is created, the processing device 3 continues to operate the screw implantation device 1, such that the motor 11 transmits power to the first rod 122 through the coupling 121, so as to drive the first rod 122 to move toward the guide hole H. In the meantime, the hollow pedicle screw T that is fixed on the first rod 122 spins at a low rotational speed and is screwed into the guide hole H in a second rotational direction (shown in FIG. 6). Furthermore, the hollow pedicle screw T spins at a second rotational speed and a second torque. Preferably, the second rotational speed is less than 300 rpm, and the second torque is greater than 5 Nm.

That is to say, the guide pin 13 creates the guide hole H under the conditions of high rotational speed and low torque, and then the hollow pedicle screw T is screwed into the guide hole H under the conditions of low rotational speed and high torque. The first rotational direction is opposite to the second rotational direction. For example, the first rotational direction is clockwise and the second rotational direction is counterclockwise. The screw implantation device 1 of the present disclosure can output different torques and rotational speeds in forward and reverse rotational directions, thereby meeting the requirements of creating the guide hole H and locking the hollow pedicle screw T.

During the process that the hollow pedicle screw T is screwed into the guide hole H, since the guide pin 13 and the hollow pedicle screw T are spaced apart from and do not interfere with each other, the guide pin 13 remains stationary. In terms of the relative position between the guide pin 13 and the hollow pedicle screw T, when the hollow pedicle screw T is locked into the guide hole H, the guide pin 13 synchronously retracts from the guide hole H relative to the hollow pedicle screw T. That is, the guide pin 13 moves in a direction opposite to an advancing direction of the hollow vertebral screw T. After the hollow pedicle screw T is locked into the guide hole H and fixed at a predetermined position, the power transmission assembly 12 is disconnected from the hollow pedicle screw T, so that the hollow pedicle screw T is separated from the first rod 122. Then, the processing device 3 controls the robot device 2 to move the screw implantation device 1 outside the surgical incision S.

By repeating steps S1 to S9, a plurality of hollow pedicle screws T can be locked at the pedicles of the patient spine. After a hollow pedicle screw T passes through the pedicle, a front end of the hollow pedicle screw T is locked into the spine, while a rear end (i.e., the U-shaped structure) of the hollow pedicle screw T is exposed for connecting with a connecting rod (not shown in the figures). Therefore, the effect of fixing vertebrae can be achieved by connecting the hollow pedicle screws T to the connecting rod.

The screw implantation device 1 provided by the present disclosure can drive the guide pin 13 spinning at a high rotational speed (e.g., above 10,000 rpm), allowing the tip 131 of the guide pin 13 to quickly drill into the bone surface to create the guide hole H at the instant of contacting bone surface, without slipping or shifting. Therefore, the position of forming the guide hole H can be more precise. Additionally, through the mechanism design that the hollow pedicle screw T is spaced apart from the guide pin 13, the hollow pedicle screw T encloses around the guide pin 13 and remains stationary, preventing the guide pin 13 from contacting and damaging other parts (i.e., non-surgical parts) of the patient's body during high-speed rotation.

In addition, in the present disclosure, the requirements of creating the guide hole H and locking the hollow pedicle screw T can be met by using a same instrument (i.e., the screw implantation device 1). There is no need to change instruments during the process of creating the guide hole H and locking the hollow pedicle screw T, and so unnecessary instrument installation steps can be omitted. Moreover, through the mechanism design of the power transmission assembly 12 (i.e., the first rod 122 and the second rod 123) of the screw implantation device 1 provided by the present disclosure, the guide pin 13 can advance and retract when the motor 11 rotates in different rotational directions. Therefore, when the hollow pedicle screw T is locked into the spine, the guide pin 13 can return into the hollow pedicle screw T, ensuring that the spine is not damaged.

As shown in FIG. 2, the screw implantation device 1 includes a torque sensor 14 and a motor encoder 15. The torque sensor 14 and the motor encoder 15 are connected to the motor 11. The torque sensor 14, also known as a torque meter, is used to detect a torque value of the motor 11. The torque sensor 14 can be a strain gauge-type torque sensor, a capacitive-type torque sensor, or a piezoelectric-type torque sensor, and the present disclosure is not limited thereto. The motor encoder 15 is used to detect a rotational speed value of the motor 11, the torque sensor 14 and the motor encoder 15 are electrically connected to the processing device 3, and the processing device 3 is used to read the rotational speed value and the torque value of the motor 11, so as to monitor operation of the screw implantation device 1.

Once the processing device 3 detects a change in the speed or torque value of the motor 11, the processing device 3 will control the screw implantation device 1 to immediately stop operation of the motor 11 and prevent bone fractures. Additionally, if the processing device 3 detects that the robot device 2 deviates from the predetermined surgical path due to external forces, the processing device 3 provides a visual or auditory warning signal through the display device 5 (e.g., a red flashing on the display screen or a specific sound from the buzzer) to alert the medical personnel. In summary, the automatic screw implantation system D of the present disclosure can immediately detect displacement of the screw implantation device 1 and provide alerts to the medical personnel through the visual or auditory signals during surgical navigation, thereby enhancing positioning reliability and navigation accuracy.

### Beneficial Effects of the Embodiment

In the automatic screw implantation system provided by the present disclosure, the motor to drives the guide pin to spin at a high rotational speed and a low torque for drilling a guide hole, and then the motor then drives the power transmission assembly to screw the hollow pedicle screw into the guide hole at a low rotational speed and a high torque. Therefore, the automatic screw implantation system combines the functions of drilling and screwing. As a result, spinal fusion surgery can be completed without changing surgical instruments, thereby simplifying the surgery procedures and enhancing the efficiency and accuracy of the surgery.

In the conventional technique, a guide hole for screwing a pedicle screw is created by manually tapping on the bone surface with a drill, which is prone to slipping on smooth bone surfaces. The screw implantation device 1 provided by the present disclosure can drive the guide pin 13 to spin at a high rotational speed (e.g., above 10,000 rpm), allowing the tip 131 of the guide pin 13 to quickly drill into the bone surface to create the guide hole H at the instant of contacting bone surface, without slipping or shifting. Therefore, the position of forming the guide hole H can be more precise. Additionally, through the mechanism design that the hollow pedicle screw T is spaced apart from the guide pin 13, the hollow pedicle screw T encloses around the guide pin 13 and remains stationary, preventing the guide pin 13 from contacting and damaging other parts (i.e., non-surgical parts) of the patient's body during high-speed rotation.

Furthermore, the existing methods for implanting the pedicle screws require exchange of instruments to separately create the guide hole H and insert the hollow pedicle screw T. In the present disclosure, the requirements of creating the guide hole H and locking the hollow pedicle screw T can be met by using a same instrument (i.e., the screw implantation device 1). There is no need to change instruments during the processes of creating the guide hole H and locking the hollow pedicle screw T, and unnecessary instrument installation steps can be omitted.

In addition, the existing pedicle screw implantation process requires pre-installation of the guide wire that may pass through the hollow pedicle screw, and the hollow pedicle screw can move to the position of the guide hole along the guide wire and for being implemented. However, the method using the guide wire cannot ensure that the guide pin or the pedicle screw does not penetrate the spine. In the present disclosure, through the mechanism design of the power transmission assembly 12 (i.e., the first rod 122 and the second rod 123) of the screw implantation device 1 provided by the present disclosure, the guide pin 13 can advance and retract when the motor 11 rotates in different rotational directions. Therefore, when the hollow pedicle screw T is locked into the spine, the guide pin 13 can return into the hollow pedicle screw T, ensuring that the spine is not damaged.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An automatic screw implantation system, comprising:
a screw implantation device including:
a power transmission assembly used to implant a hollow pedicle screw;
a guide pin passing through and arranged coaxially with the hollow pedicle screw; and
a motor dynamically coupled to the power transmission assembly and the guide pin;
wherein the motor is configured for driving the guide pin to advance and simultaneously spin at a first rotational speed and a first torque;
wherein the motor is configured for driving the power transmission assembly to control the hollow pedicle screw to spin at a second rotational speed and a second torque, the second rotational speed is smaller than the first rotational speed, and the second torque is greater than the first torque.

2. The automatic screw implantation system according to claim 1, wherein the first rotational speed is greater than 10,000 rpm, the first torque is smaller than 0.5 Nm, the second rotational speed is smaller than 300 rpm, and the second torque is greater than 5 Nm.

3. The automatic screw implantation system according to claim 1, wherein the guide pin spins in a first rotational direction, the hollow pedicle screw spins in a second rotational direction, and the first rotational direction is opposite to the second rotational direction.

4. The automatic screw implantation system according to claim 1, wherein the guide pin and the hollow pedicle screw are spaced apart from and do not interfere with each other.

5. The automatic screw implantation system according to claim 4, wherein, when the guide pin spins, the hollow pedicle screw is sleeved around the guide pin and remains stationary.

6. The automatic screw implantation system according to claim 1, wherein, when the motor is configured to drive the power transmission assembly for controlling the hollow pedicle screw to spin, the guide pin retracts relative to the hollow pedicle screw.

7. The automatic screw implantation system according to claim 1, wherein the automatic screw implantation system further includes a robot device and a processing device, the processing device is electrically connected to the robot device, the robot device is connected to the screw implantation device, and the processing device is used to control operation of the robot device.

8. The automatic screw implantation system according to claim 7, wherein the automatic screw implantation system further includes a surgical navigation module, the surgical navigation module includes a plurality of navigation markers, the plurality of navigation markers are disposed on the robot device, the screw implantation device, and a target site, such that a spatial coordinate system is established, and the processing device plans a predetermined surgical path based on the spatial coordinate system.

9. The automatic screw implantation system according to claim 8, wherein the robot device is configured to move the screw implantation device according to the predetermined surgical path, and the robot device further calibrates a posture of the screw implantation device according to the predetermined surgical path; wherein the posture includes a position and an angle of the screw implantation device.

10. The automatic screw implantation system according to claim 7, wherein the screw implantation device includes a torque sensor and a motor encoder that are connected to the motor, the torque sensor is used to detect a torque value of the motor, and the motor encoder is used to detect a rotational speed value of the motor, the torque sensor and the motor encoder are electrically connected to the processing device, and the processing device is used to read the rotational speed value and the torque value of the motor, so as to monitor operation of the screw implantation device.
